(19) United States
(12) Patent Application Publication
(10) Pub. No.: US 2005/0112187 A1
(43) Pub. Date: May 26, 2005

(54) Combination product intended for carrying out a cytotoxic treatment, in particular an antitumour treatment, in a mammal

(75) Inventor: Olivier Meyer, Kirchheim (FR)

Correspondence Address: BURNS DOANE SWECKER & MATHIS L L P, POST OFFICE BOX 1404, ALEXANDRIA, VA, 22313-1404, US

(73) Assignee: Transgene S.A., Strasbourg Cedex (FR)

(21) Appl. No.: 10/978,119

(22) Filed: Oct. 29, 2004

Related U.S. Application Data
(62) Division of application No. 09/880,038, filed on Jun. 14, 2001, now abandoned.
(60) Provisional application No. 60/246,090, filed on Nov. 7, 2000.

(30) Foreign Application Priority Data
Jun. 14, 2000 (FR) 00 07604

(51) Int. Cl. A61K 48/00; A61K 9/127; C12N 15/88

(52) U.S. Cl. 424/450; 514/44; 435/458

(57) ABSTRACT

The present invention relates to a combination product comprising at least one nucleic acid containing a sequence encoding a polypeptide or interest and at least one phospholipid of interest, for use which is simultaneous, consecutive or spread out over time, characterized in that said polypeptide and phospholipid of interest have cytotoxic activity.

The present invention relates to a combination product comprising (i) at least one nucleic acid containing a sequence encoding a polypeptide and (ii) at least one phospholipid, said polypeptide and phospholipid having cytotoxic activity, in particular antitumour activity. The present invention is particularly useful in the context of treating proliferative diseases, for example in the context of a treatment for cancer.

To date, the most encouraging results obtained in the context of antitumour treatments relate to combined treatments which associate a treatment based on chemical compounds (chemotherapy) and a treatment based on the use of radiation (radiotherapy). Besides the considerable inconveniences which this type of treatment causes for the patient, it is noted, in a large number of cases, that tumour cells, which may or may not be of the metastatic type, persist in the individual treated, possibly bringing about a relapse and therefore not allowing complete remission.

More recently, studies carried out in the cancer field have proposed adapting gene therapy protocols to antitumour therapy. In this regard, mention may be made, for example, of the studies by Meneguzzi et al. (1991, Virology, 181, 61-69) relating to immunization against tumour cells using a recombinant vaccinia vector expressing the E6 and E7 genes of type 16 human papilloma virus (see also U.S. Pat. Nos. 5,744,133 and 6,007,806) or by Leroy et al. (1998, Res. Immunol. 149, 681-684), which showed that cytokine production at tumour sites after intratumoural administration of recombinant viral vectors enables the induction of an immune response combined with inhibition of tumour growth. Mention may also be made of the results obtained using genetically modified cells capable of expressing IL2 (Roschlitz et al., 1999, Cancer Gene Ther., 6, 271-281) or viral vectors expressing a tumour antigen and an interleukin (Bizouarne et al., 1996, In “Breast cancer. Advances in Biology and Therapeutics”, F. Calvo, M. Crepin and H. Magdalenat eds., 303-308—for a review, see Zhang, 1999, Cancer Gene Therapy, 6, 113-138).

In 1997, Son (Cancer Gene Ther., 4, 391-396) proposed an alternative antitumour treatment method, combining a first treatment based on platinum (Cis-diaminedichloroplatinum II) and a second treatment by gene therapy consisting of the administration of cationic lipoplexes (i.e. complexes made of nucleic acid and cationic lipid) vehiculing the gene encoding gamma interferon.

However, the antitumour response observed in the context of these various treatments, although encouraging, does not allow a satisfactory antitumour treatment which ensures the definitive disappearance of the tumour cells while at the same time avoiding the occurrence of undesirable side effects (for example, in the case of platinum, renal, auditory, haematological or neurological toxicity). Consequently, it is desirable to have novel products and/or novel methods which make it possible to carry out antitumour treatments which are effective and easy to set up, i.e. which allow a sustained control of the tumour volume and an increase in the rate of survival of the patients treated, and which have little or no side effects other than the cytotoxic activity desired.

Alkyl lysophospholipids represent a class of antitumour drugs (U.S. Pat. No. 4,935,520), the effect of which on the apoptosis of tumour cell lines has been shown in vitro (Ruiter et al., 1999, Cancer Research, 59, 2457-2463), and more particularly in combination with treatment by irradiation (gamma radiation).

We have now identified novel combination products, the various constituents of which are chosen so as to obtain a synergistic effect of their respective cytotoxic activities and/or improved properties of said constituents. More particularly, such combination products make it possible to inhibit and/or to delay cell proliferation by inducing the specific death of the cells, in particular tumour cells, better antigen presentation and/or stimulation of the immune cells of the host organism. The present invention offers an advantageous and effective alternative to the techniques of the prior art, in particular for treating cancer in humans or in animals.

Initially, a subject of the present invention is a combination product comprising:
(i) at least one nucleic acid containing a sequence encoding a polypeptide of interest, and
(ii) at least one phospholipid of interest, characterized in that said polypeptide and phospholipid of interest have cytotoxic activity.

Such a combination product is more particularly intended for a use which is simultaneous, consecutive or spread out over time, in the context of carrying out, in a mammal, a cytotoxic treatment, for example an antitumour treatment, or in any application requiring cell death, or the control of a phenomenon of cell proliferation, for example in the case of atherogenesis or post-angioplastic restenosis.

The term “nucleic acid” is intended to denote a double-stranded or single-stranded, linear or circular, natural isolated or synthetic, DNA and/or RNA fragment which denotes a precise chain of nucleotides, which may or may not be modified, making it possible to define a fragment or a region of a nucleic acid without any size limitation. According to a preferred embodiment, this nucleic acid is chosen from the group consisting of a cDNA; a genomic DNA; a plasmid DNA; a messenger RNA; an antisense RNA; a ribozyme; a transfer RNA; a ribosomal RNA; or a DNA encoding such RNAs; a polynucleotide free of any compound facilitating its introduction into cells; a nucleic acid associated with at least one polypeptide, in particular a polypeptide of viral origin, and more particularly of adenoviral or retroviral origin, or a synthetic polypeptide; and a nucleic acid associated with a ligand.

Preferably, according to the present invention, the term “nucleic acid” denotes a recombinant vector of plasmid or viral origin. The choice of plasmids which can be used in the context of the present invention is vast. They may be cloning and/or expression vectors. In general, they are known to those skilled in the art and a number of them are commercially available, but it is also possible to construct them or modify them using genetic manipulation techniques. By way of examples, mention may be made of the plasmids derived from pBR322 (Gibco BRL), pUC (Gibco BRL), pBluescript (Stratagene), pREP4, pCEP4 (Invitrogene) or p Poly (Lathe et al., 1987, Gene 57, 193-201). Preferably, a plasmid used in the context of the present invention contains an origin of replication which ensures replication initiation in a producer cell and/or a host cell (for example, the ColE1 origin will be selected for a plasmid intended to be produced in E. coli and the oriP/EBNA1 system will be selected if self-replication of the plasmid in a mammalian host cell is desired (Lupton and Levine, 1985, Mol. Cell. Biol. 5, 2533-2542; Yates et al., Nature 313, 812-815)). It may also comprise a selection gene which makes it possible to select or identify the cells transfected (for example complementation of an auxotrophy mutation, gene encoding resistance to an antibiotic). Of course, it may comprise additional elements which improve its persistence and/or its stability in a given cell (cer sequence which promotes the persistence in monomeric form of a plasmid (Summers and Sherrat, 1984, Cell 36, 1097-1103)), sequences for integration into the cellular genome.

As regards a viral vector, it is possible to envisage a vector which is derived from a poxvirus (for example vaccinia virus, in particular MVA, canaripox), from an adenovirus, from a retrovirus, from a herpesvirus, from an alphavirus, from a foamyvirus or from an adeno-associated virus. Use will preferably be made of a nonreplicating and nonintegrating vector. In this respect, adenoviral vectors are most particularly suitable for the implementation of the present invention. However, it should be noted here that, in the context of the implementation of the present invention, the nature of the vector is relatively unimportant.

Retroviruses have the property of infecting and integrating mainly in dividing cells and, in this respect, are particularly suitable for the cancer application. A recombinant retrovirus according to the invention generally comprises the LTR sequences, an encapsidation region and the nucleotide sequence according to the invention placed under the control of the retroviral LTR or of an internal promoter, such as those described hereinafter. It may derive from a retrovirus of any origin (murine, primate, feline, human, etc.), and in particular from MoMuLV (Moloney murine leukaemia virus), MSV (murine sarcoma virus) or Friend murine retrovirus (Fb29). It is propagated in an encapsidation line capable of providing, in trans, the gag, pol and/or env viral polypeptides required for constituting a viral particle. Such lines are described in the literature (PA317, Psi CRIP GP+Am-12, etc.) The retroviral vector according to the invention can comprise modifications in particular in the LTRs (replacement of the promoter region with a eukaryotic promoter) or in the encapsidation region (replacement with a heterologous encapsidation region, for example of the VL30 type) (see French applications 94/08300 and 97/05203).

Use may also be made of an adenoviral vector which is replication-defective, i.e. lacking all or part of at least one region essential for replication, selected from the E1, E2, E4 and/or L1-L5 regions. A deletion of the E1 region is preferred. However, it may be combined with other modification(s)/deletion(s) affecting in particular all or part of the E2, E4 and/or L1-L5 regions, in so far as the defective essential functions are complemented, in trans, by means of a complementation line and/or of an auxiliary virus, in order to ensure the production of the viral particles of interest. In this respect, use may be made of vectors of the prior art, such as for example those described in international applications WO 94/28152 and WO 97/04119. By way of illustration, the deletion of the majority of the E1 region and of the E4 transcription unit is most particularly advantageous. With the aim of increasing the cloning capacities, the adenoviral vector may also lack all or part of the nonessential E3 region. According to another alternative, it is possible to use a minimum adenoviral vector retaining only the sequences essential for encapsidation, i.e. the 5′ and 3′ ITRs (Inverted Terminal Repeat) and the encapsidation region. Moreover, the origin of the adenoviral vector according to the invention may be varied from the point of view of both the species and the serotype. It may derive from the genome of an adenovirus of human or animal (for example canine, avian, bovine, murine, ovine, porcine, simian) origin or of a hybrid comprising fragments of adenoviral genome of at least two different origins. Mention may be made more particularly of the CAV-1 or CAV-2 adenovirus of canine origin, the DAV adenovirus of avian origin or the type 3 Bad adenovirus of bovine origin (Zakharchuk et al., Arch. Virol., 1993, 171-176; Spibey and Cavanagh, J. Gen. Virol., 1989, 70: 165-172; Jouvenne et al., Gene, 1987, 60: 21-28; Mittal et al., J. Gen. Virol., 1995, 76: 93-102). However, preference will be given to an adenoviral vector of human origin, preferably deriving from a serotype C adenovirus, in particular a type 2 or 5 adenovirus. An adenoviral vector according to the present invention may be generated in vitro in Escherichia coli (E. Coli) by ligation or homologous recombination (see, for example, international application WO 96/17070) or by recombination in a complementation line. The various adenoviral vectors, and also the techniques for preparing them, are known (see, for example, Graham and Prevect, 1991, in Methods in Molecular Biology, vol 7, p. 109-128; Ed: E. J. Murey, The Human Press Inc.).

The expression “nucleic acid containing a sequence encoding a polypeptide of interest” is intended to indicate that said nucleic acid comprises a gene encoding a polypeptide of interest, and elements for expressing a said gene. The term “polypeptide” is taken to mean no restriction regarding its size or its degree of glycosylation.

When the nucleic acid comprises a sequence comprising a polypeptide of interest, it should be specified that said nucleic acid comprises, in addition to the elements required to ensure the expression of said sequence after transfer into a target cell, in particular promoter sequences and/or regulatory sequences which are effective in said cell and, optionally, the sequences required for the secretion, or the expression at the surface of the target cells, of said polypeptide. The elements required for expression consist of all of the elements allowing the transcription of the nucleotide sequence into RNA and the translation of the mRNA into a polypeptide, in particular the promoter sequences and/or regulatory sequences which are effective in said cell and, optionally, the sequences required for the secretion, or the expression at the surface of the target cells, of said polypeptide. These elements may be regulatable or constitutive of course, the promoter is suitable for the vector selected and for the host cell. By way of examples, mention may be made of the eukaryotic promoters of the PGK (phosphoglycerate kinase), MT (metallothionein; Mc Ivor et al., 1987, Mol. Cell Biol., 7, 838-848), α-1 antitrypsine and CFTR genes, the promoters of the gene encoding muscle creatine kinase, actin, lung surfactant, immunoglobulins, β-actin (Tabin et al., 1982, Mol. Cell Biol., 2, 426-436) and SRα (Takebe et al., 1988, Mol. Cell Biol., 8, 466-472), the SV40 virus (simian virus) early promoter, the RSV (Rouse Sarcoma Virus) LTR, the MPSV promoter, the HSV-1 TK promoter, the CMV virus (cytomegalovirus) early promoter, the vaccinia virus promoters p7.5K, pH5R, pK1L, p28 and p11, and the E1A and MLP adenoviral promoters, or a combination of said promoters. It may also be a promoter which stimulates the expression of the gene in a tumour cell. Mention may be made in particular, of the promoters of the MUC-1 gene over-expressed in breast cancers and prostate cancers (Chen et al., 1995, J. Clin. Invest., 96, 2775-2782), CEA (for carcinoma embryonic antigen) gene overexpressed in colon cancers (Schrewe et al., 1990, Mol. Cell. Biol., 10, 2738-2748), tyrosinase gene overexpressed in melanomas (Vile et al., 1993, Cancer Res. 53, 3860-3864), ERB-2 gene overexpressed in breast cancers and cancers of the pancreas (Harris et al., 1994, Gene Therapy, 1, 170-175) and α-fetoprotein gene overexpressed in liver cancers (Kanai et al., 1997, Cancer Res., 57., 461-465). The cytomegalovirus (CMV) early promoter is most particularly preferred. It is also possible to use a promoter region which is tissue specific, in particular when the tumour to be treated is derived from a particular cell type, or which can be activated under defined conditions. The literature provides a large amount of information relating to such promoter sequences. In addition, said nucleic acid can contain at least two sequences, which may be identical or different, having transcriptional promoter activity and/or at least two sequences encoding a polypeptide of interest, which may be identical or different, and which are located, with respect to one another, contiguously or far apart, and in the same direction or in the reverse direction, provided that the function of the transcriptional promoter or the transcription of said sequences is not affected. When the nucleic acid contains at least two sequences encoding a polypeptide, it should be noted that at least one of them should encode a polypeptide of interest as defined according to the present invention (i.e. having at least cytotoxic activity); with regard to the other sequences, they may also encode such a polypeptide, or any other polypeptide which those skilled in the art will judge useful to express in the context of the techniques of the invention (for example an antigen, in particular a tumour antigen, all or part of an antibody, in particular an antibody specific for tumour antigens). Similarly, it is possible to introduce into this type of nucleic acid construct “neutral” nucleic acid sequences, or introns, which do not harm the transcription and are spliced before the translation step. Such sequences and their uses are described in the literature (WO 94/29471). Said nucleic acid may also contain sequences required for intracellular transport, for replication and/or integration, for secretion, or for transcription or translation. Such sequences are well known to those skilled in the art. Moreover, the nucleic acids which can be used according to the present invention may also be nucleic acids which are modified such that it is impossible to integrate them into the genome of the target cell, or nucleic acids which are stabilized using agents., such as for example spermine, which, in themselves, have no effect on the efficiency of the transfection. In the context of the present invention, it is possible to use all or only a portion of the nucleic acid sequence encoding the polypeptide of interest, or a derived or mutated polypeptide, provided that the function and the cytotoxic properties of this polypeptide are conserved. For the purpose of the present invention, the term “mutation” is intended to mean a deletion and/or a substitution and/or an addition of one or more nucleotides. Similarly, it is conceivable to use a sequence encoding a hybrid polypeptide originating from the fusion of the sequences encoding a polypeptide of interest according to the invention and of the sequence encoding a polypeptide of another type (for example cytotoxic polypeptide, membrane-anchoring polypeptide, secretion polypeptide).

The term “phospholipid” is intended to denote a molecule, or a combination of molecules, comprising at least one polar domain and at least one phosphorous atom. These molecules are well known to those skilled in the art (see, for example Silvius, 1993, Structure and Nomenclature. In Phospholipids Handbook. G. Cevc ed. Marcel Dekker, Inc., New York, Basle, Hong Kong, pp. 1-22). Among polar domains, mention may be made, for example, of domains derived from choline, from ethanolamine, from serine, from inositol, from glycerol or from phosphatidylglycerol. The phospholipid may also comprise an apolar domain, in particular domains derived from fatty acids, from glycerol or from steroids, and from analogues thereof. The phospholipids may be synthetic or natural, and of animal or plant origin.

The expression “compound (i.e. phospholipid or polypeptide) having at least cytotoxic activity” is intended to indicate that the compound under consideration (i.e. phospholipid or polypeptide) is capable of inducing or of activating an immune response directed specifically against a target cell, or of inhibiting the growth and/or division of such a cell. According to a preferred case, this cytotoxic activity results in the death of said cell. In a preferred embodiment of the invention, said target cell is a tumour cell (the cytotoxic activity is then termed antitumour activity). However, such cytotoxic activity may also be desired in the context of a treatment intended to correct pathological situations associated with cell proliferation, as is the case, for example, in phenomena of restenosis or of atherosclerosis (Ross, 1990, Nature, 362, 801-809; Landau et al., 1994, New Engl. J. Med., 330, 981-993). The invention also relates to such applications.

The cytotoxic activity of a given polypeptide, in particular an antitumour activity, can be evaluated in vitro by measuring cell survival, either using short term viability assays (such as, for example, the trypan blue or MTT assay) or using clonogenic survival assays (formation of colonies) (Brown and Wouters, 1999, Cancer Research, 59, 1391-1399), or in vivo by measuring tumour growth (size and/or volume) in an animal model (Ovejera and Houchens, 1981, Semin. Oncol., 8, 386-393).

According to a particularly advantageous embodiment, the phospholipid present in the combination product of the invention has a general formula: 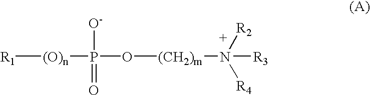
in which:
R₁ is:
   (a) either a linear or branched carbon-based chain comprising from 6 to 30 carbon atoms,
   (b) or a motif of formula: 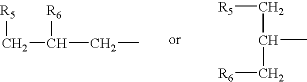
   in which R₅ represents an -A-R group, with A selected from —O—, —C(O)—, —OC(O)—, —C(O)O—, —C(S)—, —C(O)—S—, —S—, —NH— or —C(O)—NH—, and R being a linear or branched carbon-based chain comprising from 8 to 30 carbon atoms,
   and R₆ either represents a hydrogen atom or has the same meaning as R₅, with R₅ and R₆ possibly being identical or different,
and R₂, R₃ and R₄ are either hydrogen atoms or alkyl residues containing from 1 to 5 carbon atoms,
or else 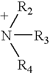
   is a cyclic amine
m is a positive integer ranging from 1 to 6 and
n is a positive integer ranging from 0 to 1.

According to a particular embodiment, R₁ is a carbon-based chain containing from 12 to 22 carbon atoms, and preferably containing 16 carbon atoms.

The carbon-based chain comprising from 6 to 30 carbon atoms represented by R₁ may be saturated or unsaturated (for example alkyl, alkenyl, alkynyl, aralkyl, etc.), and linear or branched. The alkenyl group may be a Z or E isomer. R₁, and also the R₅ and/or R₆ groups, may in addition be substituted with one or more alkyl (C₁-C₅ in particular), hydroxy, mercapto, amino, oxo, carbamoyl, carboxy, halogen, C₃-C₇ cycloalkyl, C₃-C₇ cycloalkenyl, aryl (for example phenoxy, tolyl, phenyl, etc.), fluorine, etc. groups.

By way of examples of R₁, mention may be made of C₆-C₃₀ alkyl radicals [for example, n-dodecyl, n-tridecyl, n-tetradecyl, 3,7,11-trimethyldodecyl, n-pentadecyl, n-heptadecyl, n-octadecyl, n-eicosyl, n-docosyl, 3,7-dimethyloctyl(1-octyl)nonyl and 3,7,11,15-tetramethylhexadecyl]; C₆-C₃₀ alkenyl radicals [for example, 8-tridecenyl(δ8), 3,7,11-trimethyl-2,6,10-dodecatrienyl, 8-tetradecenyl(δ8), 8,11-tetradecadienyl(δ8,11), 8-heptadecenyl(δ8), 2-octadecenyl, 9-octadecenyl(oleyl), 9,15-octadecadienyl, 9,12,15-octadecatrienyl, 8,11,14-heptadecatrienyl (δ8,11,14), 8,11-octadecadienyl(δ8,11), 4,7,10,13-nonadecatetraenyl(δ4,7,10,13), phythyl, 3,7,11,15-tetramethyl-2,6,10,14-hexadecatetraenyl, 3,7,11,15-tetramethyl-2,4,6,10,14-hexadecapentaenyl, 12-(2,3-cyclopentenyl)dodecyl, 12-(2,3-cyclopentenyl)-5-dodecenyl, 11-hydroxy-8-heptadecenyl, 3,7-dimethyl-9-(2,6,6-trimethyl-1-cyclohexen-1-yl)-2,4,6,8-nonatetraenyl and 4,7,10,13-nonadecatetraenyl]; C₆-C₃₀ alkynyl radicals (for example, 9-octadecynyl, 9,15-octadecadiynyl, heptadecan-8-ynyl and 4-decynyll; C₆-C₃₀ aralkyl radicals [for example, 15-(4-n-butylphenyl)pentadecyl, omega-(p-tolyl)heptadecyl, 6-(4-n-pentylphenyl)hexadecyl and 15-phenyl-pentadecyl], and 15-(4-n-butylphenoxy)pentadecyl or 6-(4-n-pentylphenoxy)hexadecyl. According to an advantageous case of the invention, R₁ is a C₁₀-C₃₀ alkyl radical.

The R₂, R₃ and R₄ groups may also be substituted with one or more groups such as those mentioned earlier on for R₁. By way of examples, mention will be made of the cases according to which R₂, R₃ and R₄ are substituted with a hydroxycarbonyl, a C₁-C₃ alkoxycarbonyl, a hydroxyl, a cyano group or a C₁-C₃ alkoxy.

When the group associated with N is a cyclic amine, mention will be made, by way of example, of the cases for which it represents a pyridino, oxazolo, thiazolo, pyridazino, quinolino or isoquinolino radical. These radicals may also be substituted with groups such as C₁-C₅ alkyl (for example methyl, ethyl, etc.), hydroxyl, hydroxyethyl, aminoethyl, amino (imino), carbamoyl or ureido groups. The cyclic amine mentioned also includes the cases in which any one of the R₂, R₃ and R₄ radicals forms a ring with the quaternary amine and the remaining radical is a C₁-C₄ alkyl radical (for example methyl, ethyl), for example N-ethylmorpholino or N-methylpiperidino.

According to an advantageous embodiment, the invention relates to the case for which n=1, m=2 and R₂, R₃ and R₄ are methyl radicals; in an entirely preferred case, R₁ is an unbranched alkyl radical comprising 16 carbon atoms.

In addition, because of the presence of reactive functions (for example amino, hydroxy, etc. functions), such chemical molecules may be substituted directly or via an arm, such as for example a heterodifunctional reagent (for example SPDP or SMCC) or a reagent which has been made functional (for example PEG). Such reagents are well documented in the literature (Mattson et al., 1993, Mol. Biol. Reports, 17, 167-183). The element for substitution may be one of those widely described in the literature, for example a labelling molecule (see, for example, U.S. Pat. No. 4,711,955) making it possible to visualize the distribution of said lipid after it has been administered in vitro or in vivo; a molecule allowing targeting of cells (ligands) or cellular anchoring; or an element facilitating penetration into cells, reduction of endosomes (JTS1 peptides for example, Gottchalk et al., 1996, Gene Therapy, 3, 448-457) or intracellular transport. These molecules may consist, entirely or partly, of a sugar, of a glycol, of a peptide (for example GRP, Gastrin Releasing Peptide), of an oligonucleotide, of a lipid (in particular C₂-C₂₂ lipids), of a hormone, of a vitamin, of an antigen, of an antibody (or fragments thereof), of a specific membrane-bound receptor, of a ligand capable of reacting with a cellular anti-ligand, of a fusogenic peptide, of a nuclear localization (NLS) peptide, or a combination of such molecules, for example galactosyl residues for targeting the asialoglycoprotein receptor at the surface of hepatocytes, the INF-7 fusogenic peptide derived from the HA-2 subunit of the influenza virus (Plank et al. 1994, J. Biol. Chem., 269, 12918-12924), or the NLS signal of the T antigen of the SV40 virus (Lanford and Butel, 1984, Cell, 37, 801-813) or of the Epstein Barr virus (Ambinder et al., 1991, J. Virol., 65, 1466-1478). Several studies using in particular the phage display technique have made it possible to identify peptide sequences which can be used as substituents according to the invention and which allow the specific targeting of certain cells, such as for example brain or kidney cells (Pasqualini et al., 1996, Nature 380, 364-366), lung cells (Romanczuk et al., 1999, Human Gene Therapy, 10, 2615-2626), skin cells or pancreatic cells (Rajotte et al., 1998, J. Clin. Invest., 102, 430-437), or cells from certain types of tumour (Pasqualini et al., 1997, Nature Biotechnology, 15, 542-546; Christiano et al., 1996, Cancer Gene Therapy, 3, 4-10; Croce et al., 1997, Anticancer Res., 17, 4287-4292; Gottschalk et al., 1994, Gene Ther., 1, 185-191; Park et al., 1997, Adv. Pharmacol., 40, 399-435). The entire contents of these articles form part of the present application. By way of example, mention may be made of the HWGF motif, which is a ligand for the metalloproteinases involved in tumour growth, in angiogenesis and in the formation of metastases (Koivunen et al., 1999, Nature Biotechnology, 17, 768-774).

The substituted phospholipids of the invention can be easily obtained according to techniques which are within the scope of those skilled in the art, more particularly using chemical coupling groups, for example chemical groups such as trifluoroacetyl, Fmoc (9-fluoroenylmethoxycarbonyl) or BOC (tert-butyl-oxycarbonyl) on an amine (Greene T. W. and Wuts P. G. M., 1991, Protective groups in organic synthesis. Ed. J. Wiley & Sons, Inc. New York).

According to a particular case of the invention, the phospholipid of the invention is in the form of a salt combined with an anion, such as the chlorine, bromine or iodine ions, with an ion of an alkali metal (for example Na⁺, K⁺) or with an ion of an alkaline-earth metal (for example Ca²⁺, Mg²⁺).

The phospholipid of formula A, and derivatives thereof, can be prepared by any suitable methods or by those indicated in U.S. Pat. No. 4,935,520, the content of which is incorporated into the present application by way of reference. Certain derivatives of the phospholipid presented in formula A and also usable in the context of the invention are also described in WO 90/15807.

In a preferred case of the invention, the phospholipid present in the combination product is in a zwitterionic form, i.e. its positive/negative charge ratio is zero.

According to one embodiment of the invention, the polypeptide of interest which is encoded by the sequence included in said nucleic acid is chosen from cytokines, polypeptides having chemoattractant activity (i.e. chemokines), proteins encoded by a gene termed “suicide gene”, anti-angiogenic protein factors and polypeptides having an activity for activating cellular apoptosis.

Cytokines are molecules which are naturally produced subsequent to an antigenic stimulation or to an inflammatory reaction (Gillis and Williams, 1998, Curr. Opin. Immunol., 10, 501-503), the usefulness of which in the context of treating certain cancers has been shown in particular by Oettger (Curr. Opin. Immunol., 1991, 3, 699-705). According to this first variant of the invention, the polypeptide of interest will preferably denote a cytokine chosen from α-, β- and γ-interferon, interleukins, and in particular IL-2, IL-4, IL-6, IL-10 or IL-12, tumour necrosis factors (TNFs)- and colony stimulating factors (for example GM-CSF, C-CSF and M-CSF).

According to a preferred embodiment, said cytokine is selected from interleukin-2 (IL-2) and gamma interferon (γ-IFN). Interleukin-2 is in particular responsible for the proliferation of activated T lymphocytes, and for the multiplication and activation of cells of the immune system (for the nucleic acid sequence see in particular FR 85 09480). γ-IFN activates phagocytic cells and increases the expression of class I and class II major histocompatibility complex surface antigens (for the nucleic acid sequence see in particular FR 85 09225). These nucleic acid sequences are incorporated into the application by reference.

According to a particular embodiment, the combination product according to the invention is characterized in that it comprises at least two sequences, carried by one or more distinct nucleic acids, encoding all or part of interleukin-2 (IL-2) and all or part of gamma interferon (γ-IFN).

According to a second variant of the invention, the polypeptide of interest is a polypeptide having chemoattractant activity (i.e. chemokines). Chemokines constitute a subclass of the cytokine family. They differ from the other cytokines by their chemoattractant property, in particular during natural chemotactic processes, and especially natural processes of attraction of cells of the immune system towards the tissues in which the inflammation or the infection lies, and also by their anti-angiogenic properties.

Chemokines are low molecular weight (between 8 and 10 kd) proteins which are small in size (from 70 to 80 amino acids), and the amino acid sequences of which exhibit a low degree of homology (ranging from 10 to 70% depending on the chemokines considered) making it possible to define, to date, approximately 50 different chemokines. These chemokines can, however, be subdivided into 4 major families, relative to the position of the cysteine residues which they contain. The a family, the N-terminal end of which comprises 2 cysteines separated by a single amino acid (chemokines of the IL-8, NAP-2, GCP-2 type) and the ∃ family, the N-terminal end of which comprises 2 adjacent cysteines (chemokines of the RANTES, MIP1, MCP1 type), are the most well characterized (Horuk, R., 1994, Trends Pharmacol. Sci., 15, pages 159-165; Murphy, P. M., 1994, Annu. Rev. Immunol., 12, pages 593-633).

In the context of the present invention, the preferred chemokine is the chemokine of the MIP1 type, and more particularly selected from the group consisting of the MIP1α and MIP1β chemokines, the properties of which have been demonstrated by Wolpe et al. (1988, J. Exp. Med. 167, 570-581).

MIP1α, the nucleic acid and peptide sequences of which are described in Obaru et al. (1986, J. Biochem., 99, 885-894), the content of which is incorporated into the present application by reference, is produced by T lymphocytes and monocytes. It enables chemoattraction of eosinophils and T lymphocytes during respiratory tract infections; and of monocytes and neutrophils during rheumatoid arthritis, digestive tract inflammations or meningitis of bacterial origin. In addition, it inhibits the proliferation of haematopoietic precursors.

MIP1β, the nucleic acid and peptide sequences of which are described in Brown et al. (1989, J. Immunol., 142, 679-68), the content of which is incorporated into the present application by reference, is also produced by T lymphocytes and monocytes. It exercises its chemoattractant properties on monocytes and neutrophils in cases of osteoarthritis and bacterial meningitis. Like MIP1α, it inhibits the proliferation of haematopoietic precursors.

There are natural variants of said MIP1α and MIP1β proteins, which are known to those skilled in the art and which bear, for example, the names GOS19, LD78, pAT464, TY5 (mouse) or SISA (mouse) for MIP1α, or pAT744, Act-2, G-26, H-400 (mouse) or hSISγ (mouse) for MIP1β. In the particular case of MIP1β, the sequence corresponding to Act-2 (Lipes et al., 1988, PNAS, 85, 9704-9708), the content of which is incorporated herein by reference, will, for example, be chosen.

According to a third variant of the invention, the polypeptide of interest is a polypeptide encoded by a gene termed “suicide gene”. Several studies have made it possible to identify polypeptides which are not toxic in themselves, but which have catalytic enzymatic properties capable of transforming an inactive substance (prodrug), for example a nucleoside or a nucleoside analogue, into a subtance which is highly toxic for the cell, for example a modified nucleoside which may be incorporated into the DNA chain or RNA chain undergoing elongation, with, as a result, particularly the inhibition of cell division or cellular dysfunction leading to the death of the cell containing such polypeptides. The genes encoding such polypeptides are termed “suicide genes”. Many suicide gene/prodrug pairs are currently available. Mention may be made more particularly of the pairs:
herpes simplex virus type 1 thymidine kinase (HSV-1 TK) and acyclovir or ganciclovir (GCV) (Caruso et al., 1993, Proc. Natl. Acad. Sci. USA 90, 7024-7028; Culver et al., 1992, Science 256, 1550-1552; Ram et al., 1997, Nat. Med. 3, 1354-1361):
rat cytochrome p450 and cyclophosphophamide (Wei et al., 1994, Human Gene Therapy 5, 969-978);
Escherichia coli (E. coli) purine nucleoside phosphorylase and 6-methylpurine deoxyribonucleoside (Sorscher et al., 1994, Gene Therapy 1, 233-238):
E. coli guanine phosphoribosyl transferase and 6-thioxanthine (Mzoz and Moolten, 1993, Human Gene Therapy 4, 589-595) and.
cytosine deaminase (CDase) and 5-fluoro-cytosine (5FC).

According to an advantageous case, the invention relates to the case according to which said polypeptide of interest has at least one enzymatic activity selected from thymidine kinase activity, purine nucleoside phosphorylase activity, guanine, uracil or orotate phosphoribosyltransferase activity, and cytosine deaminase activity.

More particularly, CDase is an enzyme involved in the metabolic pathway for pyrimidines, through which exogenous cytosine is transformed, via hydrolytic deamination, into uracil. CDase activities have been demonstrated in prokaryotes and lower eukaryotes (Jund and Lacroute, 1970, J. Bacteriol. 102, 607-615; Beck et al., 1972, J. Bacteriol. 110, 219-228; De Haan et al., 1972, Antoine van Leeuwenhoek 38, 257-263; Hoeprich et al., 1974, J. Inf. Dis. 130, 112-118; Esders and Lynn, 1985, J. Biol. Chem. 260, 3915-3922) but they are absent in mammals (Koechlin et al., 0.1966, Biochem. Pharmacol. 15, 435-446; Polak et al., 1976, Chemotherapy 22, 137-153). The FCY1 gene of Saccharomyces cerevisiae (S. cerevisiae) and the codA gene of E. coli, which encode, respectively, the CDase of these two organisms, are known and their sequences are published (EP 402 108; Erbs et al., 1997, Curr. Genet. 31, 1-6; WO 93/01281). CDase also deaminates a cytosine analogue, 5-fluorocytosine (5-FC), to 5-fluorouracil (5-FU), which is a highly cytotoxic compound, in particular when it is converted to 5-fluoro-UMP (5-FUMP). Cells which lack CDase activity, due either to an inactivating mutation of the gene encoding the enzyme or to their natural deficiency for this enzyme (for example mammalian cells) are resistant to 5-FC (Jund and Lacroute, 1970, J. Bacteriol. 102., 607-615; Kilstrup et al., 1989, J. Bacteriol., 171, 2124-2127). On the other hand, it has been demonstrated that it is possible to transmit 5-FC sensitivity to mammalian cells into which the sequence encoding a CDase activity has been transferred (Huber et al., 1993, Cancer Res. 53, 4619-4626; Mullen et al., 1992, Proc. Natl. Acad. Sci. USA 89, 33-37; WO 93/01281). In addition, in this case, the neighbouring nontransformed cells also become sensitive to 5-FC (Huber et al., 1994, Proc. Natl. Acad. Sci. USA 91, 8302-8306). This phenomenon, termed bystander effect, is due to the excretion, by the cells expressing the CDase activity, of 5-FU which intoxicates the neighbouring cells by simply diffusing through the cell membrane. This passive diffusion property of 5-FU constitutes an advantage with respect to the tk/GCV reference system, for which the bystander effect requires contact with the cells which express tk (Mesnil et al., 1996, Proc. Natl. Acad. Sci. USA 93, 1831-1835). Consequently, this effect constitutes an additional asset of the use of CDase in the context of gene therapy, in particular anticancer gene therapy.

However, 5-FC sensitivity varies a great deal depending on the cell lines. Low sensitivity is observed, for example, in PANC-1 (carcinoma of the pancreas) and SK-BR-3 (breast adenocarcinoma) human tumour lines transduced with a retrovirus expressing the coda gene of E. coli (Harris et al., 1994, Gene Therapy 1, 170-175). This undesirable phenomenon may be explained by the absence or poor endogenous conversion of the 5-FU formed by the enzymatic action of the CDase, to cytotoxic 5-FUMP. This step, which is normally carried out in mammalian cells by orotate phosphorybosyltransferase (Peters et al., 1991, Cancer 68, 1903-1909), may be absent in certain tumours and thus make gene therapy based on CDase ineffective. In prokaryotes and lower eukaryotes, uracil is transformed into UMP through the action of uracil phosphoribosyl-transferase (consequently exhibiting UPRTase activity). This enzyme also converts 5-FU to 5-FUMP. Thus, furl mutants of the yeast S. cerevisiae are resistant to high concentrations of 5-FU (10 mM) and of 5-FC (10 mM) since, in the absence of UPRTase activity, the 5-FU, originating from the deamination of the 5-FC by the CDase, is not transformed into cytotoxic 5-FUMP (Jund and Lacroute, 1970, J. Bacteriol. 102, 607-615). The upp and FUR1 genes encoding the UPRTase of E. coli and of S. cerevisiae, respectively, have been cloned and sequenced (Andersen et al., 1992, Eur. J. Biochem. 204, 51-56; Kern et al., 1990, Gene 88, 149-157).

According to one embodiment of the present invention, the polypeptide of interest has UPRTase activity, which means that said polypeptide is capable of converting uracil, or a derivative thereof, into a monophosphate analogue, and in particular 5-FU into 5-FUMP.

The UPRTase to which the present invention refers may be of any origin, in particular prokaryotic, fungal or yeast origin. By way of illustration, the nucleic acid sequences encoding the UPRTases of E. coli (Anderson et al., 1992, Eur. J. Biochem 204, 51-56), of Lactococcus lactis (Martinussen and Hammer, 1994, J. Bacteriol. 176, 6457-6463), of Mycobacterium bovis (Kim et al., 1997, Biochem Mol. Biol. Int 41, 1117-1124) and of Bacillus subtilis (Martinussen et al., 1995, J. Bacteriol. 177, 271-274) can be used in the context of the invention. However, use of a yeast UPRTase, and in particular that encoded by the FUR1 gene of S. cerevisiae, the sequence of which disclosed in Kern et al. (1990, Gene 88, 149-157) is introduced herein of reference, is most particularly preferred. By way of indication, the sequences of the genes and those of the corresponding UPRTases can be found in the literature and the specialized data banks (SWISSPROT, EMBL, Genbank Medline, etc.).

Moreover, application PCT/FR99/00904 describes an FUR1 gene lacking 105 nucleotides on the 5′ side of the coding portion, enabling the synthesis of a UPRTase deleted of the first 35 residues in the N-terminal position and starting at the methionine at position 36 in the native protein. The expression product of the mutant gene, denoted FUR1)105, is capable of complementing an furl mutant of S. cerevisiae. In addition, the truncated mutant has a UPRTase activity which is greater than that of the native enzyme. Thus, according to a particularly advantageous embodiment, the encoded polypeptide according to the invention is a deletion mutant of a native UPRTase. The deletion is preferably located in the N-terminal region of the UPRTase of origin. It may be total (concern all of the residues of said N-terminal region) or partial (concern one or more residues which may be continuous or discontinuous in the primary structure). In general, a polypeptide consists of N-terminal, central and C-terminal portions, each representing approximately one third of the molecule. For example, since the UPRTase of S. cerevisiae has 251 amino acids, its N-terminal portion consists of the first 83 residues starting at the methionine, termed initiating methionine, located at the first position of the native form. As regards the UPRTase of E. coli, its N-terminal portion covers positions 1 to 69.

In addition, patent applications WO 96/16183 and PCT/FR99/00904 describe the use of a fusion protein encoding an enzyme with two domains having CDase activity and UPRTase activity, and demonstrate that the transfer of a coda::upp or FCY1::FUR1 or FCY1::FUR1Δ105 hybrid gene carried by an expression plasmid increases the 5-FC sensitivity of transfected B16 cells. The protein and nucleic acid sequences described in those two applications are incorporated into the description of the present application. According to this embodiment, the polypeptide is a polypeptide fused in frame with at least a second polypeptide. Although the fusion may take place at any site on the first polypeptide, the N- or C-terminal ends are preferred, and in particular the N-terminal end. Fusion of the CDase and UPRTase activities makes it possible to improve the sensitivity of the target cells to 5-FC and to 5-FU.

Those skilled in the art are capable of cloning the CDase or UPRTase sequences using the published data, of carrying out possible mutations, of assaying the enzymatic activities of the mutant forms in an acellular or cellular system according to the technology of the art or by following the protocol indicated in application PCT/FR99/00904, and of fusing, in particular in frame, the polypeptides having CDase and UPRTase activity, and consequently all or part of the corresponding genes. Hybrid polypeptides as described in patent applications WO 96/16183 and PCT/FR99/00904 are incorporated into the application by reference.

According to another variant, the polypeptide of interest is an anti-angiogenic protein factor. Angiogenesis is the process responsible for the formation of new capillaries from the already existing vascular network. This complex process is finely regulated in healthy tissues by the balance of the effects of many angiogenic and anti-angiogenic factors. However, in certain pathological conditions, and in particular in the formation of a tumour, this process is disturbed: the angiogenic factors override the anti-angiogenic factors, which allows considerable vascularization of tumours and, as a result, their rapid development and/or the appearance of metastases. For this reason, in the context of the present invention, an anti-angiogenic factor is considered to be a cytotoxic agent, in particular a cytotoxic antitumour agent. Among the various anti-angiogenic factors which are known at the present time, mention may be made, in particular, of angiostatin, endostatin, platelet factor PF4, thrombospondin-1, PRP (for proliferin related protein), VEGI (for vascular endothelial growth inhibitor), metalloproteases and urokinase.

Finally, the polypeptide of interest may be a polypeptide having activity for activating cellular apoptosis, and more particularly the p53 protein. p53 is a nuclear phosphoprotein which is involved in particular in controlling the expression of proteins involved in the cell cycle (Ozbun et al., 1995, Adv. Cancer Res. 66, 71-141; Selter et al., 1994, Int. J. Biochem. 26, 145-154), and which contributes to many cellular processes related to the stability of the genome and to cellular apoptosis (Harris et al., 1996, J. Natl. Cancer Inst. 88, 1442-1445; Kastan et al., 1991, Cancer Res. 51, 6304-6311; Kuerbitz et al., 1992, PNAS, 89, 7491-7495). The p53 gene has been identified and sequenced. The sequence of the cDNA is described in Matlashewski et al. (1984, EMBO J., 3, 3257-3262), and that of the protein is described in Lamp (1986, Mol. Cell Biol., 6, 1379-1385). Similarly, natural and functional polymorphic variants have been identified for which certain amino acids are replaced with others without, however, affecting p53 function. Moreover, many mutations which can result in a loss of the function of this protein have been described in the literature relating to cancer (Holstein et al, 1991, Science, 253, 49-53; Levine et al., 1991, Nature, 351, 453-456). For example, Baker et al. (1989, Science, 244, 217) have noted that, in more than 70% of colorectal tumours, the function of this p53 gene is lost. In the context of the present invention, it is possible to use the entire nucleic acid sequence encoding the p53 polypeptide or only a portion of this polypeptide, or a derived or mutated polypeptide, provided that the function of p53 is conserved. Such sequences are well known to those skilled in the art and it is possible to refer, for example, to Matlashewski et al. (1984, EMBO J., 3, 3257-3262), Prives et al. (1994, Cell, 78, 543-546) or Chen et al. (1996, Gene and Deve., 10, 2438-2451), the contents of which are incorporated into the present application. Given the properties of the p53 polypeptide as a transcriptional transactivator (Farmer et al., 1992, Nature, 358, 83-86) or as a polypeptide capable of interacting with other proteins (Harris, 1996, Carcinogenesis, 17, 1187-1198), the p53 activity can be measured by analysing the cell cycle arrest in the G1/S and G2/M phase, the induction of apoptosis, the suppression of oncogene-induced cellular transformation or the inhibition of angiogenesis.

The sequences encoding the polypeptides of interest of the invention can be easily obtained by cloning, by PCR or by chemical synthesis according to the conventional techniques in use. They may be native genes or genes derived from the latter by mutation, deletion, substitution and/or addition of one or more nucleotides. Moreover, their sequences are widely described in the literature which can be consulted by those skilled in the art.

According to a particular embodiment of the invention, the combination product is characterized in that it also comprises:
(iii) a substance which associates with nucleic acids and/or
(iv) a substance which associates with the phospholipids of interest.

The expression “substance which associates with nucleic acids” is intended to denote a substance, or a combination of several substances, which makes it possible in particular to improve transfection efficiency and/or the stability of a vector, particularly of a vector of plasmid origin, and/or the protection of said vector in vivo against the immune system of the host organism (Rolland A, Critical reviews in Therapeutic Drug Carrier System, 15, (1998), 143-198). These substances associate with nucleic acids by electrostatic, hydrophobic, cationic, covalent or preferably noncovalent interaction. Such substances are widely documented in the literature which is accessible to those skilled in the art (see, for example, Felgner et al., 1987, Proc. West. Pharmacol. Soc. 32, 115-121; Hodgson and Solaiman, 1996, Nature Biotechnology, 14, 339-342; Remy et al., 1994, Bioconjugate Chemistry 5, 647-654). By way of illustration, but without limitation, they may be cationic polymers or cationic lipids, but also liposomes, nuclear or viral proteins, or neutral lipids. These substances can be used alone or in combination. Examples of such compounds, and also of methods for measuring their capacity to improve transfection efficiency and/or the stability of a given vector, are in particular available in patent applications WO 98/08489, WO 98/17693, WO 98/34910, WO 98/37916, WO 98/53853, EP 890362 or WO 99/05183. They may in particular be lipid substances such as DOTMA (Felgner et al., 1987, PNAS, 84, 7413-7417), DOGS or Transfectam™ (Behr et al., 1989, PNAS, 86, 6982-6986), DMRIE or DORIE (Felgner et al., 1993, Methods, 5, 67-75), DC-CHOL (Gao and Huang, 1991, BBRC, 179, 280-285), DOTAP™ (McLachlan et al., 1995, Gene Therapy, 2, 614-622) or Lipofectamine™.

Advantageously, these cationic lipids are selected from the cationic lipids of formula. (see EP 901 463): 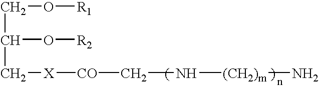
in which:
   R₁ and R₂, which may be identical or different, are linear or branched C₆-C₂₃ alkyls or C₆-C₂₃ alkenyls, or linear or branched C₆-C₂₃ alkylcarbonyls or C₆-C₂₃ alkenylcarbonyls,
   X is O, S, S(O) or —NR₃, R₃ is a hydrogen atom or C₁-C₄ alkyl,
   n is a positive integer between 1 and 6,
   m is a positive integer between 1 and 6, and when n>1, m may vary within the same molecule.

The substance (iii) may also be a cationic polymer, such as for example polyamidoamine (Haensler and Szoka, Bioconjugate Chem. 4 (1993), 372-379), a “dendrimer” polymer (WO 95/24221), polyethyleneimine or polypropyleneimine (WO 96/02655), chitosan, a polyamino acid such as polylysine (U.S. Pat. No. 5,595,897 or FR 2 719 316); a polyquaternary compound; protamine; polyimines; polyvinylamines; polycationic polymers substituted with DEAE, such as pullulans or celluloses; polyvinyl-pyridine; polymethacrylates; polyacrylates; polyoxethanes; poly(thiodiethylaminomethylethylene) (P(TDAE)); polyhistidine; polyornithine; poly-p-aminostyrene; copolymethacrylates (for example copolymers of HPMA; N-(2-hydroxypropyl)methacrylamide); the compounds described in U.S. Pat. No. 3,910,862, the complexes of DEAE polyvinylpyrrolide with methacrylate, dextran, acrylamide, polyimines, albumin, 1-dimethylaminomethyl methacrylate and polyvinylpyrrolidonemethylacrylaminopropyltrimethylammonium chloride; telomeric compounds (patent application EP 98401471.2). However, this list is not exhaustive and other known cationic polymers may be used to obtain the nucleic acid complexes of the invention. In addition, these cationic polymers and lipids may be fluorinated (see, for example, WO 98/34910).

The expression “substance (iv) which associates with the phospholipids” is intended to denote in particular a molecule, or a combination of molecules, capable of integrating into a structure vehiculing the phospholipid of interest. Among these structures, mention may be made, for example, of liposomes, micelles or nanoparticles. Among the substances (iv) which associate with the phospholipids of interest, mention may be made, for example, of molecules capable of integrating into liposomes, in particular lipids (see, for example, Paternostre et al., 1996, Liposomes: preparation and membrane protein reconstitution. In Manual on membrane lipids. R. Prasad ed. Springer-Verlag, Berlin, Heidelberg pp. 202-247) and proteins. As regards lipids, polar lipids, nonpolar lipids (for example carotenoids or steryl esters), certain steroids, such as for example sterols, phospholipids or glycolipids can, for example, be envisaged. As regards proteins, mention may be made, for example, of membrane-bound proteins, transmembrane proteins or proteins with phosphatidylinositol-type anchoring, these proteins possibly being glycosilated. According to an advantageous embodiment of the invention, the substance (iv) may enable the labelling of the structure vehiculing the phospholipid of interest, the targeting of cells or cellular anchoring, facilitate penetration into the cells, the reduction of endosomes or cellular transport, or increase the plasmatic half-life of the liposome (see, for example, Chonn et al., 1995, Curr. Opin. Biotechnol., 6, 698-708).

35. The invention also relates to the case according to which said combination product also contains an adjuvant (v) selected from neutral, zwitterionic or negatively charged lipids. These neutral, zwitterionic or negatively charged lipids may, for example, be selected from the group comprising natural phospholipids of animal or plant origin, such as phosphatidylcholine, phosphocholine, phosphatidylethanolamine, sphingomyelin, phosphatidylserine, phosphatidylinositol, ceramide or cerebroside, and analogues thereof; synthetic phospholipids which generally, but not exclusively, comprise two identical fatty acid chains, such as dimyristoylphosphatidylcholine, dioleoylphosphatidylcholine, dipalmitoylphosphatidylcholine, distearoylphosphatidylcholine, phosphatidylethanolamine (PE) and phosphatidylglycerol, and analogues thereof; phosphatidylchloline, cardiolipin, phosphatidylethanolamine, mono-, di- or triacylglycerol, and alpha-tocopherol, and analogues thereof; phosphatidylglycerol, phosphatidic acid or a similar phospholipid analogue; cholesterol, glycolipids, fatty acids, sphingolipids, prostaglandins, gangliosides, niosomes or any other natural or synthetic amphiphile.

According to a preferred case, said adjuvant is selected from cholesterol, dioleoylphosphatidylethanolamine (DOPE) and derivatives thereof.

According to an advantageous embodiment of the invention, said nucleic acid (i), said substance (iii), said phospholipid (ii) and, optionally, said adjuvant (v) form a complex. Such a complex results from the association of the various compounds with one another, for example by electrostatic, hydrophobic, cationic, covalent or preferably noncovalent interaction.

Such complexes can be defined with reference to various characteristics. By way of examples, mention may be made of:
the theoretical charge ratio (+/− or P/N) which represents the ratio between the number of positive charges and the number of negative charges provided by all of the constituents of said complex. In calculating this theoretical charge ratio, it is presumed that all the cationic groups and all the anionic groups are
   for example, be selected from the group comprising natural phospholipids of animal or plant origin, such as phosphatidylcholine, phosphocholine, phosphatidylethanolamine, sphingomyelin, phosphatidylserine, phosphatidylinositol, ceramide or cerebroside, and analogues thereof; synthetic phospholipids which generally, but not exclusively, comprise two identical fatty acid chains, such as dimyristoylphosphatidylcholine, dioleoylphosphatidylcholine, dipalmitoylphosphatidylcholine, distearoylphosphatidylcholine, phosphatidylethanolamine (PE) and phosphatidylglycerol, and analogues thereof; phosphatidylchloline, cardiolipin, phosphatidylethanolamine, mono-, di- or triacylglycerol, and alpha-tocopherol, and analogues thereof; phosphatidylglycerol, phosphatidic acid or a similar phospholipid analogue; cholesterol, glycolipids, fatty acids, sphingolipids, prostaglandins, gangliosides, niosomes or any other natural or synthetic amphiphile.

According to a preferred case, said adjuvant is selected from cholesterol, dioleoylphosphatidylethanolamine (DOPE) and derivatives thereof.

According to an advantageous embodiment of the invention, said nucleic acid (i), said substance (iii), said phospholipid (ii) and, optionally, said adjuvant (v) form a complex. Such a complex results from the association of the various compounds with one another, for example by electrostatic, hydrophobic, cationic, covalent or preferably noncovalent interaction.

Such complexes can be defined with reference to various characteristics. By way of examples, mention may be made of:
the theoretical charge ratio (+/− or P/N) which represents the ratio between the number of positive charges and the number of negative charges provided by all of the constituents of said complex. In calculating this theoretical charge ratio, it is presumed that all the cationic groups and all the anionic groups are ionized. Generally, an excess of positive charges in the complex facilitates the binding of said complex to the cell surface, which is negatively charged. The amounts and the concentration of the constituents of a given complex will be adjusted as a function of their respective molecular mass and of their number of positive/negative charges. According to a preferred embodiment, the theoretical charge ratio (+/− or P/N) of the complex present in the combination product ranges between 0.05 and 20, preferably between 0.1 and 15, and more especially between 0.5 and 10;
the nucleic acid (i) concentration. Preferably, this concentration ranges from 10 μg/ml to 5 000 μg/ml, and more particularly from 100 μg/ml to 2 000 μg/ml;
the structure of the nucleic acid, which, according to a preferred embodiment, will have at least 80%, preferably 90%, and more preferably 95% of the nucleic acids (i) in a supercoiled form;
when the complex contains a substance (iii) and an adjuvant (v), said complex can also be defined by the molar ratio between these elements. Advantageously, the ratio between (iii) and (v) ranges between 0.1 and 10, and preferably between 2 and 5;
the complex can also be characterized by its mean diameter, which is preferably less than 2 μm, ranges between 20 and 800 nm, more particularly between 50 and 500 nm, advantageously between 75 and 200 nm, and entirely preferably is equal to approximately 100 nm. The mean diameter of the complex can be selected for optimal use in certain applications. This diameter can be measured using a large number of techniques including, but not being limited to, the technique known under the name “dynamic laser light scattering” (photon correlation spectroscopy, PCS)”, and also other techniques known to those skilled in the art (see, Washington, Particle Size Analysis in Pharmaceutics and other Industries, Ellis Horwood, New York, 1992, 135-169). It should be noted here that methods for separating complexes of the invention as a function of their size exist and can be used to select complexes with a specific diameter. Reference may, for example, be made to methods such as extrusion, sonication, micro-fluidization, exclusion/diffusion chromatography, gradient separation, electrophoresis and ultracentrifugation, particularly through a membrane with defined pores.

According to an advantageous embodiment, the phospholipid (ii) is incorporated into a said complex formed between a nucleic acid (i) and a substance (iii), possibly optionally combined with an adjuvant (v). In this precise case, the molar ratio between the phospholipid (ii) and the substance (iii), and possibly the adjuvant (v), ranges between 0.1% and 60%, advantageously between 10% and 50%, and preferably between 20% and 40%.

However, the invention also relates to a combination product in which said phospholipid is not in a complex with said nucleic acid (i). In this particular case, said phospholipid of interest (ii) is preferably associated with a substance (iv). Preferably, this association with the substance (iv) consists of a liposome which makes it possible to vehicle the phospholipid of interest. This type of liposome is known to those skilled in the art.

Mention may be made, in particular, of liposomes prepared from cholesterol, alkyl lysophospholipids and diacetyl phosphate (Zeisig et al. 1994, Anticancer research, 14, 1785-1790). According to a preferred embodiment of the invention, these liposomes will in particular consist of cholesterol. The liposome which vehicles the phospholipid of interest may also vehicle other substances of interest (see, for example, Chonn et al., 1995, Curr. Opin. Biotechnol., 6, 698-708, the content of which is inserted herein by way of reference). Among these other substances of interest, mention may be made in particular of drugs having cytotoxic activity (for example amphotericin B or all-trans-retinoic acid), recombinant proteins, and in particular those having cytotoxic activity, or nucleic acids.

The invention also relates to a combination product as described above, characterized in that it is formulated in a vehicle which is acceptable from a pharmaceutical point of view. Such a support is preferably isotonic, hypotonic or weakly hypertonic, and has a relatively low ionic strength, such as, for example, a sucrose solution. Furthermore, such a support may contain any solvent, or aqueous or partially aqueous liquid, such as nonpyrogenic sterile water. The pH of the formulation is, in addition, adjusted and buffered in order to satisfy the requirements of use in vivo. The formulation may also include a diluent, an adjuvant or an excipient which is acceptable from a pharmaceutical point of view, and also solubilization agents, stabilization agents and/or preservatives. For injectable administration, a formulation in aqueous, nonaqueous or isotonic solution is preferred. It can be provided in a single dose or in multiple doses, in a liquid or dry (powder, lyophilizate, etc.) form which can be reconstituted extemporaneously with a suitable diluent.

According to a particular embodiment of the invention, said combination product also comprises amounts which are acceptable from a pharmaceutical point of view of a prodrug capable of being transformed into a cytotoxic molecule by a polypeptide having at least cytotoxic activity. Such a prodrug will in particular be selected from the group consisting of acyclovir or ganciclovir (GCV), cyclophosphophamide, 6-methylpurine deoxyribonucleoside, 6-thioxanthine, cytosine or a derivative thereof, or uracil or a derivative thereof. In addition, when said prodrug is 5-fluorocytosine (5FC) or 5-fluorouracil (5-FU), said combination product can also comprise one or more substances which potentiate the cytotoxic effect of the 5-FU. Mention may be made, in particular, of drugs which inhibit the enzymes of the pathway for de novo biosynthesis of pyrimidines (for example those cited hereinafter), drugs such as Leucovorin (Waxman et al., 1982, Eur. J. Cancer Clin. Oncol. 18, 685-692) which, in the presence of the product of 5-FU metabolism (5-FdUMP), increases the inhibition of thymidylate synthase, which causes a decrease in the pool of dTMP required for replication and, finally, drugs such as methotrexate (Cadman et al., 1979, Science 0.250, 1135-1137) which, by inhibiting dihydrofolate reductase and increasing the pool for incorporation of PRPP (phosphoribosylpyrophosphate) causes an increase in 5-FU in the cellular RNA.

The combination product of the invention may also contain a substance selected from the group comprising, for example, chloroquine, protic compounds, such a propylene glycol, polyethylene glycol, glycerol, ethanol, 1-methyl L-2-pyrrolidone, and derivatives thereof, aprotic compounds, such as for example dimethyl sulphoxide (DMSO), diethyl sulphoxide, di-n-propyl sulphoxide, dimethyl sulphone, sulpholane, dimethylformamide, dimethylacetamide, tetramethylurea, acetonitrile, or derivatives thereof (see EP 890 362), cytokines, particularly interleukin-10 (IL-10) (WO 99/56784), hyaluronidase (WO 98/52853) and nuclease inhibitors (WO 99/56784), such as for example actin G. In another embodiment of the invention, this substance can be a salt, and preferably a cationic salt, such as for example magnesium (Mg²⁺) (EP 998945) and/or lithium (Li⁺). In this case, the amount of ionic substance in the complex of nucleic acids of the invention advantageously ranges between 0.1 mM and approximately 100 mM, and preferably between 0.1 mM and approximately 10 mM.

Another subject according to the invention consists of a complex comprising:
(i) at least one nucleic acid containing a sequence encoding a polypeptide of interest,
(ii) at least one phospholipid of interest,
(iii) a substance which associates with nucleic acids,
(v) optionally, an adjuvant,
characterized in that the said phospholipid of interest (ii) has the characteristics and properties described above, and more particularly in that it has a general formula: 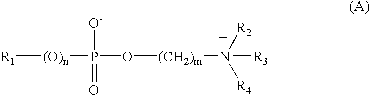
in which:
   R₁ is:
      (a) either a linear or branched carbon-based chain comprising from 6 to 30 carbon atoms,
      (b) or a group of formula: 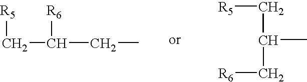
      in which R₅ represents an -A-R group, with A selected from —O—, —C(O)—, —OC(O)—, —C(O)O—, —C(S)—, —C(O)—S—, —S—, —NH— or —C(O)—NH—, and R being a linear or branched carbon-based chain comprising from 6 to 30 carbon atoms,
      and R₆ either represents a hydrogen atom or has the same meaning as R₅, with R₅ and R₆ possibly being identical or different,
   and R₂, R₃ and R₄ are either hydrogen atoms or alkyl residues containing from 1 to 5 carbon atoms,
   or else 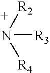
   is a cyclic amine
   m is a positive integer ranging from 1 to 6 and
   n is appositive integer ranging from 0 to 1.

The elements of the description regarding the nucleic acids (i), the phospholipid (ii), the substance which associates with nucleic acids (iii) and the adjuvant are also applicable to the characterization of such a complex.

Advantageously, depending on the nature of the vector used, the combination product of the invention will comprise in its ready-to-be-administered form:
when the vector is of plasmid origin, from 0.01 to 100 mg of DNA, preferably between 0.05 and 10 mg, and entirely preferably from 0.5 to −5 mg;
when the vector is of viral origin, between 10⁴ and 10¹⁴ pfu (plaque-forming units), advantageously between 10⁵ and 10¹³ pfu, and preferably between 10⁶ and 10¹² pfu.

Another subject according to the invention consists of the use of a combination product or of a complex, as described above, for preparing a medicinal product intended to treat the human or animal body, and more particularly intended for antitumour and/or antimetastatic treatment, intended especially to inhibit the growth, or cause the rejection, of a tumour, or the death of an infected cell. According to another case, the treatment carried out will consist in controlling the cell proliferation observed in the case of damage from atherosclerosis or from restenosis.

As indicated above, the combination product of the invention, which comprises at least one nucleic acid (i) containing a sequence encoding a polypeptide of interest and at least one phospholipid of interest (ii), can be implemented in the context of a use which is simultaneous, consecutive or spread out over time. The term “simultaneously” refers to a co-administration. In this case, the two components (i) and (ii) can be mixed prior to the administration, or can be administered at the same time to the host organism or cell. It is also possible to administer them consecutively, i.e. one after the other, regardless of which component of the combination product according to the invention is administered first. Finally, use may be made of a method of administration which is spread out over time or intermittent, and which is stopped and restarted at intervals which may or may not be regular, for one and/or other of the two components. It is indicated that the routes and sites of administration of the two components may be different. According to a particularly preferred embodiment, the nucleic acid of interest (i) and the phospholipid (ii) are present in one and the same complex as described above.

In the case of repeat injections, of one and/or other of the components (i) and (ii), optionally mixed with the substances (iii), (iv) and/or the adjuvant (v), the time interval between the injections is not critical and can be easily defined by those skilled in the art. An interval of 10 min to 72 h, advantageously of 30 min to 48 h, preferably of 1 to 24 h, and entirely preferably of 1 to 6 h can be recommended.

According to the invention, many routes of administration can be envisaged. Mention may be made, for example, of the systemic, intragastric, subcutaneous, intracardiac, intramuscular, intravenous, intraperitoneal, intratumoral, intranasal, intra-pulmonary or intratracheal route. For the latter three embodiments, administration by aerosol or instillation is advantageous. Entirely preferably, the administration of the combination product or of the complex of the invention is carried out intratumorally or peritumorally, i.e. into an accessible tumour, around its edge, or into a blood vessel connected to the organ affected or to the tumour.

Among the conceivable applications, mention may be made of breast cancers, cancers of the uterus (in particular those induced by papilloma viruses), prostate cancers, lung cancers, bladder cancers, liver cancers, colon cancers, and cancers of the pancreas, of the stomach, of the oesophagus, of the larynx, of the central nervous system and of the blood (lymphomas, leukaemia, etc.). It is also useful, in the context of cardiovascular diseases for example, to inhibit or delay the proliferation of the smooth muscle cells of the vascular wall (restenosis).

The invention also extends to a method for treating diseases by gene therapy, characterized in that a combination product or a complex according to the invention is administered to a host cell or organism needing such a treatment. According to one particular case, this treatment consists of separate administrations of, firstly, the nucleotide sequence (i) and, secondly, the phospholipid of interest (ii) according to the invention. In this particular case, said phospholipid will preferably be associated with a substance (iv) as described above. However, according to an entirely advantageous embodiment, said nucleic acid sequence (i) and said phospholipid of interest (ii) are administered concomitantly, preferably in the form of a complex comprising at the very least the nucleic acid (i) and said phospholipid (ii).

When said treatment uses a sequence encoding a polypeptide having UPRTase activity it may be advantageous to also administer a second sequence capable of expressing a second polypeptide having CDase activity. In the latter case, the administration of the UPRTase and CDase sequences can be simultaneous or consecutive, the order of administration being of no importance. According to such an embodiment, the treatment method will also comprise an additional step according to which amounts, which are acceptable from a pharmaceutical point of view, of a prodrug, advantageously of a cytosine analogue, and in particular of 5-FC, will be administered to the host cell or organism. By way of illustration, a dose of 50 to 500 mg/kg/day may be used, with a preference for. 200 mg/kg/day. This prodrug can be administered according to standard practice, this administration being prior to, concomitant with, or subsequent to, that of the combination product of the present invention. Oral administration is preferred. It is also possible to administer a single dose of prodrug or doses repeated for a time sufficiently long to allow the production of the toxic metabolite in the host cell or organism.

According to an advantageous embodiment of the invention, the use or the treatment method of the invention is combined with a second treatment of the patient by surgery (in particular by partial or total ablation of the tumour), by radiotherapy or by chemotherapy. In this particular case, the treatment according to the invention is carried out prior to, concomitant with, or subsequent to, said second treatment. Preferably, this treatment will be carried out subsequent to said second treatment.

Similarly, the efficiency of the intracellular transfer of the nucleic acid (i) can be advantageously facilitated, for example, by combination with electroporation treatment (Vicat et al., 2000, Human Gene Therapy, 11, 909-916), or treatment intended to modify the permeability of the blood vessels in which the administration is carried out (WO 98/58542), or by any other means described in the literature.

The invention also relates to the use of a phospholipid of interest, or of a derivative thereof, for preparing a complex as described in the invention, in particular intended for gene therapy applications.

Finally, the invention relates to the use of a complex as described earlier, and in particular of such a complex containing a phospholipid of interest, for simultaneously introducing a nucleic acid sequence (i) and such a phospholipid (ii) into a target cell, with the aim, for example, of inducing the death of said target cell.

The aim of the following examples is to illustrate the various subjects of the present invention and, consequently, they are not limiting in nature.

### DESCRIPTION OF THE FIGURES

FIG. 1: Study of the cytotoxicity of complexes containing the plasmid pTG11236, the cationic lipid pcTG201 and cholesterol, in the presence or in the absence of HPC, on RENCA kidney carcinoma cells.

FIG. 2: In vivo measurement of the luciferase activity in mouse tumours transfected with complexes containing the plasmid pTG11236, the cationic lipid pcTG201, HPC or DOPE.

FIG. 3: In vivo tumor growth after intra tumoral injection of complex comprising pcTG201, cholesterol, HPC in presence or absence of plasmid pTG14387.

FIG. 4: In vivo tumor growth after intra tumoral injection of complex comprising pcTG201, cholesterol and plasmid pTG14387 with or without HPC.

FIG. 5: In vivo tumor growth after intra tumoral injection of plasmid pTG14387, or HPC or a combination of HPC and plasmid pTG14387.

FIG. 6: Mice survival after intratumoral injection of plasmid pTG14387, or HPC or a combination of HPC and plasmid pTG14387.

### EXAMPLES

### Example 1

### Study of the Cytotoxicity of the Complexes Containing HPC

Complexes containing a plasmid pTG11236 (5739 base pairs) containing a sequence encoding luciferase placed downstream of the cytomegalovirus early promoter sequence, a cationic lipid (pcTG201, Nazih et al., 2000, Synlett., 5, 635-636), cholesterol (Sigma) and, optionally, hexadecylphosphocholine (HPC, Sigma) were prepared by sonication and then extrusion (see patent application FR 97/02420). The complexes used in this example are characterized by a diameter of 200 to 400 nm, an N/P ratio=5, a pcTG201/cholesterol molar ratio=1/1 for the complexes which do not contain HPC, or a pcTG201/HPC/cholesterol molar ratio=1/2/2 for the complexes which contain HPC.

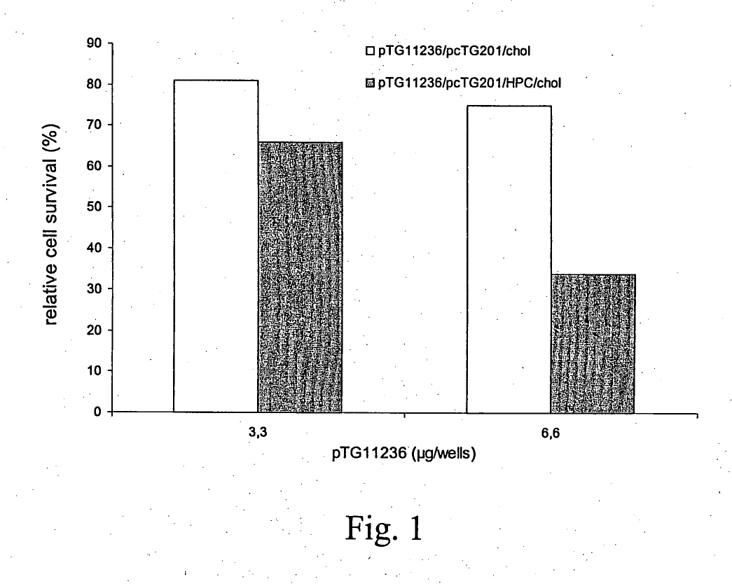

The RENCA kidney carcinoma cells were cultured in wells (96/plate) containing modified Eagle medium (DMEM) in the presence of 10% of calf serum (Gibco). Various amounts of complexes (corresponding to an amount of plasmid of 3.3 pg to 6.6 μg) were deposited onto the cells (70-80% confluent) for 48 h. After this period, the medium was removed and the cells were washed several times with a PBS buffer. A cytotoxicity assay was carried out by means of the assay using MTT tetrazolium [bromine 3-(4,5-dimethylthiazol-2-yl)-2,5-diphenyltetrazolium] (Twentyman and Luscombe, 1987, Br. J. Cancer, 56, 279-285). The results obtained are given in FIG. 1. The amount of complexes deposited is proportional to the amount of pTG11236 introduced into each well. These results indicate that, unlike the complexes which do not contain HPC, the complexes which contain HPC make it possible to bring about the death of the RENCA tumour cells.

### Example 2

### In Vivo Transfection of Mouse Tumour Cells

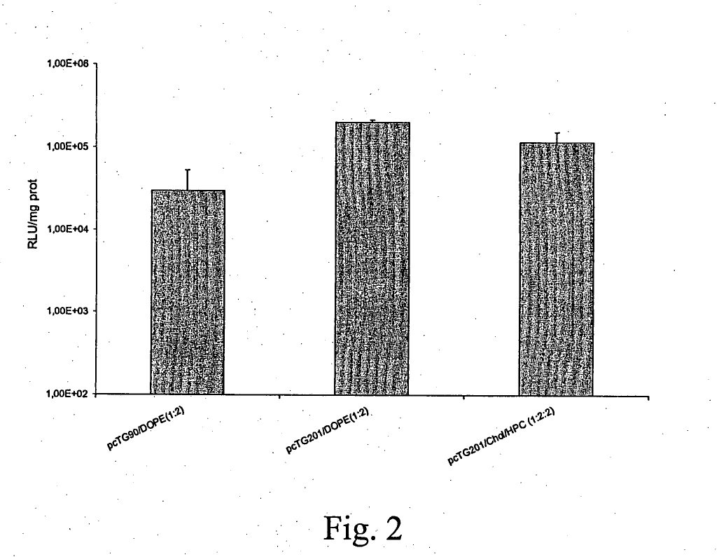

RENCA cells are injected under the skin of 8-week old B6D2 female mice (5×10⁴ cells per animal). Eleven days after the injection of the cells, palpable tumours appear. Compositions containing complexes containing the plasmid pTG11236, the lipid pcTG201 and HPC or DOPE (N/P=0.5, pcTG201/DOPE or pcTG201/HPC molar ratio=1/1) are then directly injected into the tumour (60 μg of pTG11236 injected per tumour). The mice are sacrificed 24 h after injection of the complexes and the tumours are extracted and ground in a buffer suitable for assaying luciferase activity (luciferase determination assay, Promega). A protein assay. (BCA assay, Pierce) is carried out in parallel, in order to standardize the values for relative light units (RLU) per mg of protein in the tumour. The results obtained are given in FIG. 2. These results indicate that the tumour cells injected with complexes which contain HPC express as much luciferase as the tumour cells injected with complexes which do not contain HPC, thus showing that the presence of HPC in complexes does not inhibit their transfection property.

### Example 3

### Effect of Complexes Comprising HPC on in Vivo Tumor Growth

Tumor bearing mice prepared as described in example 2 are intratumoraly injected five times (with three day-time interval between subsequent injection) with 50 μl of a composition comprising complexes made with
pcTG201, cholesterol and the plasmid pTG14837 (4557 bp, comprising a sequence coding for a cytotoxic polypeptide: IL2) or,
pcTG201, cholesterol, HPC and an empty plasmid or,
pcTG201, cholesterol, HPC and pTG14837
   24 μg DNA is injected each time (N/P=0.5, pcTG201/chol/HP=1/1/2 or pcTG201/chol=1/1). Tumor volume is measured with a caliper by determining the two perpendicular diameters and the depth of the tumor (14 mice are injected per group).

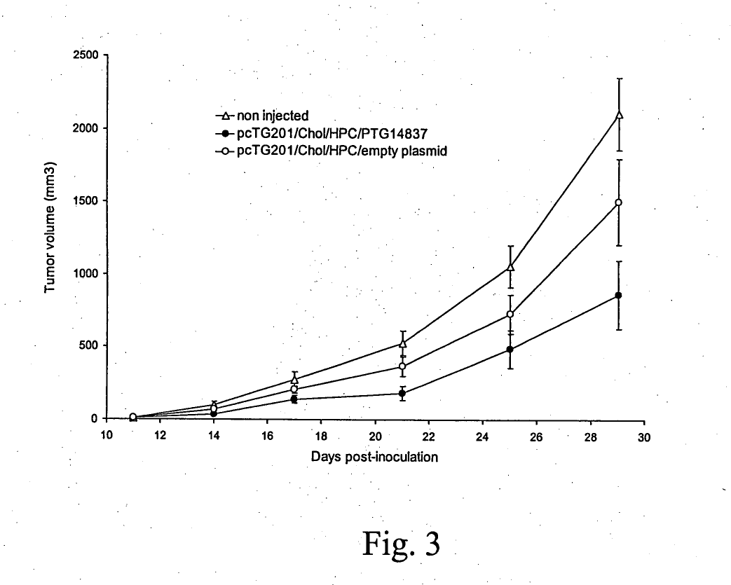

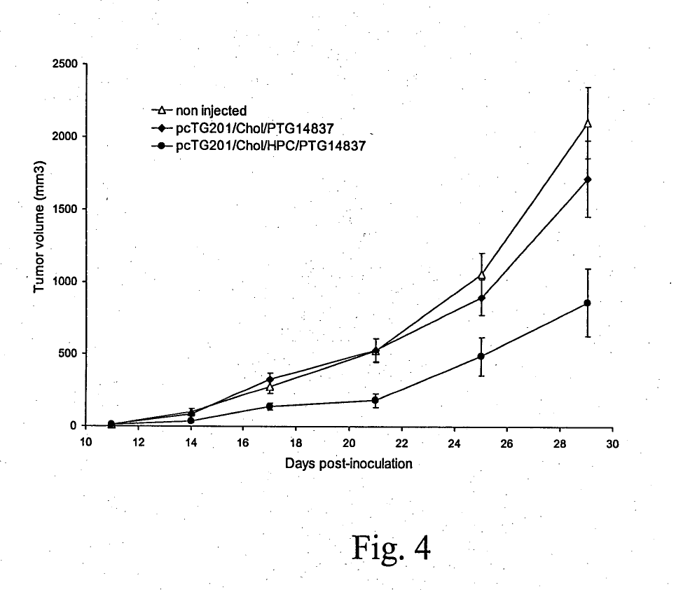

Results are depicted in FIGS. 3 and 4. They show a slower tumor growth after injection with complexes comprising a combination of HPC and pTG14387 compared to injection with complexes comprising only HPC or pTG14387.

These results clearly show the synergistic effect on tumor growth of HPC and the nucleic acid comprising the sequence coding a cytotoxic polypeptide (i.e. IL2).

### Example 4

### Effect of Compositions Comprising HPC and a Nucleic Acid Comprising the Sequence Coding for IL2 on Mouse Survival and Tumor Growth

B6D2 female mice are subcutaneously inoculated in the middle of the flank with 3 10⁵ RENCA cells. After 11-12 days, 50 μl of a composition comprising 10 μg of pTG14387 plasmid or 0,2% HPC or a combination of both in a 5% glucose solution are intratumoraly injected (nine mice are injected per group). Tumor volume is measured as previously described.

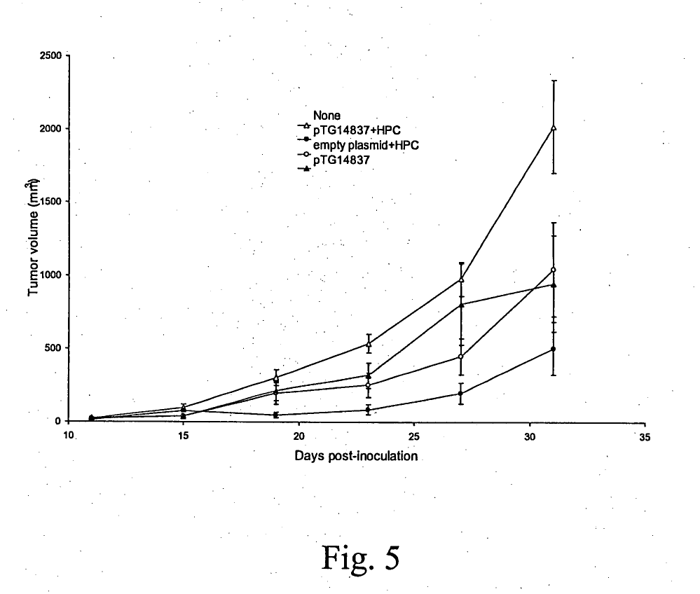

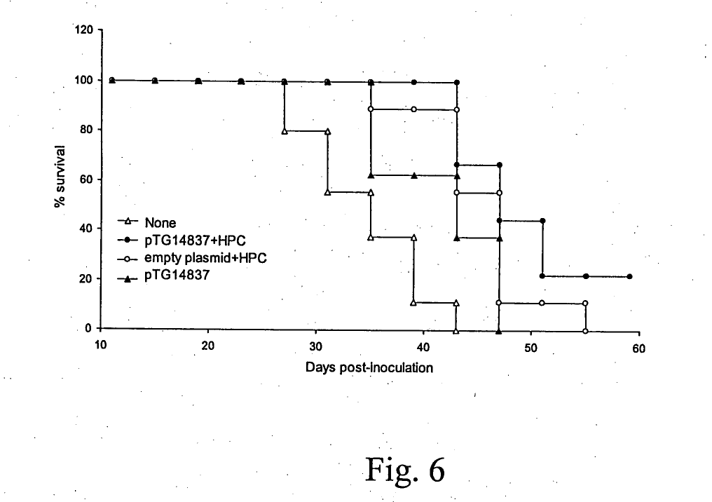

Results are depicted in FIGS. 5 and 6. They show a slower tumor growth after injection with the combination of pTG14387 and HPC compared to injection with pTG14387 or HPC alone. This decrease of tumor growth is also correlated with long term survival in 22% of the mice. These results confirmed the synergistic effects of HPC and the nucleic acid comprising the sequence coding a cytotoxic polypeptide, here IL2.

1-26. (canceled)

27. A method for treating a human or animal body comprising administering an effective amount of a combination product comprising:
(i) at least one nucleic acid containing a sequence encoding a polypeptide of interest, and
(ii) at least one phospholipid of interest, for use which is simultaneous, consecutive or spread out over time, characterized in that said polypeptide and phospholipid of interest have cytotoxic activity and said phospholipid of interest has a general formula: 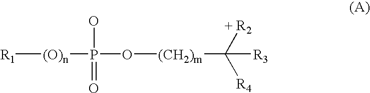
in which:
R₁ is either a linear or branched carbon-based chain comprising from 6 to 30 carbon atoms,
R₂, R₃ and R₄ are either hydrogen atoms or alkyl residues containing from 1 to 5 carbon atoms,
m is a positive integer ranging from 1 to 6 and
n is a positive integer ranging from 0 to 1 for preparing a medicinal product intended for treating the human or animal body.

28. The method of claim 27, wherein R₁ is a linear or branched carbon-based chain comprising from 12 to 22 carbon atoms.

29. The method of claim 28, wherein R₁ is a linear or branched carbon-based chain comprising 16 carbon atoms.

30. The method of claim 27, wherein R₁ is an alkyl, alkenyl, alkynyl or aralkyl residue.

31. The method of claim 27, wherein n=1, m=2 and R₂, R₃ and R₄ are methyl residues.

32. The method of claim 27, wherein R₁ is an alkyl residue comprising 16 carbon atoms.

33. The method of claim 27, wherein said polypeptide of interest is selected from the group consisting of cytokines, proteins encoded by a suicide gene, antiangiogenic protein factors, polypeptides having chemoattractant activity and polypeptides having activity for activating cellular apoptosis.

34. The method of claim 33, wherein said polypeptide of interest is a cytokine selected from the group consisting of alpha, beta and gamma interferon, interleukins, tumour necrosis factors and colony stimulating factors.

35. The method of claim 34, wherein said cytokine is interleukin-2 (IL-2) or gamma interferon (γ-IFN).

36. The method of claim 27, wherein the combination product also comprises:
(iii) a substance which associates with nucleic acids and/or
(iv) a substance which associates with the phospholipid of interest.

37. The method of claim 36, wherein said substance (iii) is a cationic lipid or a cationic polymer.

38. The method of claim 36, wherein said substance (iv) is a lipid capable of integrating into a liposome.

39. The method of claim 27, wherein said combination product also contains an adjuvant (v) selected from the group consisting of neutral, zwitterionic and negatively charged lipids.

40. The method of claim 39, wherein said adjuvant (v) is selected from the group consisting of cholesterol, dioleoylphosphatidylethanolamine (DOPE) and derivatives thereof.

41. The method of claim 36, wherein said nucleic acid (i), said substance (iii), said phospholipid (ii) and, optionally, said adjuvant (v) form a complex.

42. The method of claim 41, wherein the ratio between the number of positive charges and the number of negative charges of the elements forming said complex ranges between 0.05 and 20.

43. The method of claim 42, wherein said complex has a diameter of between 20 and 800 nm.

44. The method of claim 27, wherein said nucleic acid (i) is a recombinant vector of plasmid or viral origin.

45. The method of claim 27, wherein it is formulated in a vehicle which is acceptable from a pharmaceutical point of view.

46. The method of claim 27, wherein the treatment is an antitumour and/or antimetastatic treatment.

46. The method of claim 27, wherein said combination product is administered intratumorally or peritumorally.
